(19) United States
(12) Patent Application Publication
(10) Pub. No.: US 2010/0087780 A1
(43) Pub. Date: Apr. 8, 2010

(54) Wire Guide having Variable Flexibility and Method of Use Thereof

(75) Inventors: Kurt J. Tekulve, Ellettsville, IN (US); Thomas A. Osborne, Bloomington, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 12/245,161

(22) Filed: Oct. 3, 2008

(51) Int. Cl. A61M 25/092; A61M 25/09

(52) U.S. Cl. 604/95.01; 604/528

(57) ABSTRACT

The present invention generally relates to a medical surgical device and specifically a wire guide for percutaneous placement within a body lumen. The flexibility of the wire guide may be varied while it is in place within the body lumen. One embodiment of the wire guide includes and a distal tip positioned at the distal end of the coil. A plurality of core members are positioned side by side within the lumen. Methods of using the wire guide are also provided.

### TECHNICAL FIELD

The present invention generally relates to a medical surgical device and specifically a wire guide for percutaneous placement within a body lumen. The flexibility of the wire guide may be varied while it is in place within the body lumen.

### BACKGROUND

Wire guides are commonly used in vascular procedures, such as angioplasty procedures, diagnostic and interventional procedures, percutaneous access procedures, or radiological and neuroradiological procedures in general, to introduce a wide variety of medical devices into the vascular system. For example, wire guides are used for advancing intraluminal devices such as stent delivery catheters, balloon dilation catheters, atherectomy catheters, and the like within body lumens. Typically, the wire guide is positioned inside the inner lumen of an introducer catheter. The wire guide is advanced out of the distal end of the introducer catheter into the patient until the distal end of the wire guide reaches the location where the interventional procedure is to be performed. After the wire guide is inserted, another device such as a stent and stent delivery catheter is advanced over the previously introduced wire guide into the patient until the stent delivery catheter is in the desired location. After the stent has been delivered, the stent delivery catheter can then be removed from a patient by retracting the stent delivery catheter back over the wire guide. The wire guide may be left in place after the procedure is completed to ensure easy access if it is required.

Conventional wire guides include an elongated wire core with one or more tapered sections near the distal end to increase flexibility. Generally, a flexible body such as a helical coil or tubular body is disposed about the wire core. The wire core is secured to the flexible body at the distal end. In addition, a torquing means can be provided on the proximal end of the core member to rotate, and thereby steer a wire guide having a curved tip, as it is being advanced through a patient's vascular system.

A major requirement for wire guides and other intraluminal guiding members, is that they have sufficient stiffness to be pushed through the patient's vascular system or other body lumen without kinking. However, they must also be flexible enough to pass through the tortuous passageways without damaging the blood vessel or any other body lumen through which they are advanced. Efforts have been made to improve both the strength and the flexibility of wire guides to make them more suitable for their intended uses, but these two properties tend to be diametrically opposed to one another in that an increase in one usually involves a decrease in the other.

For certain procedures, such as when delivering stents around challenging take-off, tortuosities, or severe angulation, substantially more support and/or vessel straightening is frequently needed from the wire guide. Wire guides that provide improved support over conventional wire guides have been commercially available for such procedures. However, such wire guides are in some instances so stiff they can damage vessel linings when being advanced.

In other instances, extreme flexibility is required as well. For example, when branched or looped stents are to be delivered to a branched vascular region, it is beneficial to insert the wire guide from the branch where a stent is to be located. However, the stent may need to be introduced and guided from a separate branch. In this situation, the wire guide is inserted into the patient's vascular system near the desired stent location and a grasping device is inserted in the branch from which the stent will be introduced. The wire guide may be advanced back along the branch to provide the grasping device access to the distal end of the wire guide. However, the wire guide should be extremely flexible to allow grasping and manipulation of the wire guide without damaging the tissue around the bifurcation formed by the luminal branch. Further, the wire guide should be extremely kink resistant to avoid damaging the wire guide as it is grasped. After the wire guide is retrieved by the grasping device, the stent may be delivered over the wire guide to the desired location. However, available wire guides are not designed to provide the flexibility required to cross up and over the bifurcation of the luminal branch and yet also provide the stiffness required to aid in the insertion of the stent.

In view of the above, it is apparent that there exists a need for an improved design for a wire guide.

### BRIEF SUMMARY

One aspect provides a wire guide having variable flexibility. In one embodiment, the wire guide includes a coil defining a lumen, a distal tip positioned at the distal end of the coil and a plurality of core members positioned side by side within the lumen. At least one of the core members is attached to the distal tip and extends from the distal tip to the proximal end of the coil. The wire guide may also include a band positioned around the core members and holding the core members together. In one embodiment, at least one of the core members can be moved axially with respect to the coil, and can move proximally and away from the distal tip. In another embodiment, the core members can have a square or rectangular cross section.

In yet another embodiment, the wire guide also includes a handle attached to the proximal end of the coil. The handle includes a plurality of adjustment members, one of which is attached to one of the core members and is movable axially to vary the axial position of that core member. The handle may also include graduated markings indicating the axial position of the core member.

In another embodiment, at least one of the core members includes stainless steel, a stainless steel alloy, platinum, palladium, a nickel-titanium alloy or combinations thereof. In yet another embodiment, the coil is a multifialar coil.

Another aspect provides a method of varying the flexibility of a wire guide positioned within a body lumen of a patient. The method includes inserting the distal end of a wire guide as disclosed above into the body lumen and varying the axial position of the one of the plurality of core members.

### BRIEF DESCRIPTION OF THE DRAWINGS

FIGS. 1(a)-(c) are partial cross sections along the length of illustrative wire guides.

FIGS. 2(a)-(b) are partial cross sections along the length of one embodiment of an illustrative wire guide.

FIG. 3 is a traverse cross section of one embodiment of an illustrative wire guide.

FIG. 3 is a traverse cross section of another embodiment of an illustrative wire guide.

FIG. 4 is a traverse cross section of yet another embodiment of an illustrative wire guide.

FIG. 5 is a traverse cross section of another embodiment of an illustrative wire guide.

FIG. 6 is a traverse cross section of yet another embodiment of an illustrative wire guide.

FIG. 7 is a traverse cross section of another embodiment of an illustrative wire guide.

### DETAILED DESCRIPTION

In accordance with an embodiment of the present invention, a wire guide system includes a wire guide having a mechanism allowing the flexibility of the wire guide to be variable while the wire guide is in place within a body lumen of a human or animal patient (“patient”).

The terms proximal and distal are used herein to refer to portions of a wire guide. As used herein, the term “distal” is defined as that portion of the wire guide closest to the end of the wire guide inserted into the patient's body lumen. The term “proximal” is defined as that portion of the wire guide closest to the end of the wire guide that is not inserted into the patient's body lumen. The terms distally and proximally are used herein to refer to directions along an axis joining the proximal and distal portions of the wire guide (“axial direction”). For example, proximal movement is movement towards the proximal portion of the wire guide. Distal movement is movement towards the distal portion of the wire guide.

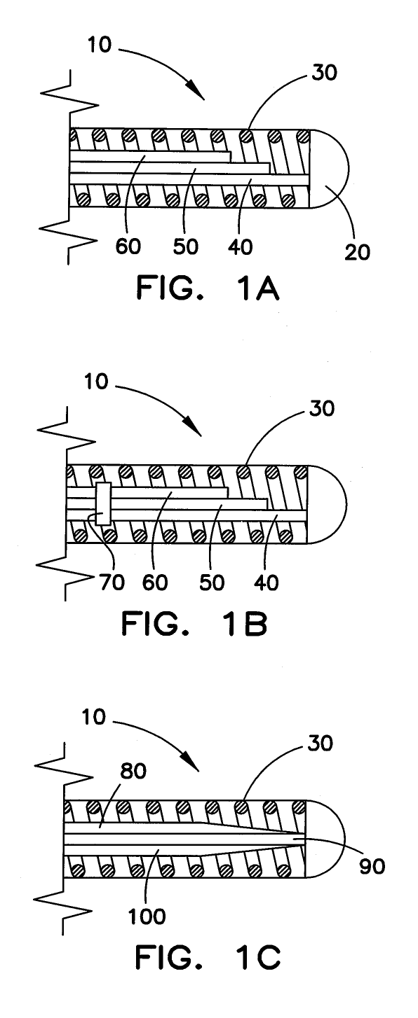

Reference is now made to FIGS. 1(a)-(c). FIG. 1(a) illustrates a partial view of one embodiment of a wire guide 10 incorporating a mechanism allowing for the flexibility of the wire guide to be varied while it is in place within the body lumen of a patient. Wire guide 10 includes coil 30 extending from the proximal portion of the wire guide to distal tip 20. Core members 40, 50 and 60 are positioned within the lumen of coil 30. FIG. 1(a) shows core member 40 extending distally to distal tip 20. Core member 50 is positioned such that its distal end is positioned proximally of the distal end of core member 40, while core member 60 is positioned such that its distal end is positioned proximally of the distal end of core member 50.

In one embodiment, core members 50 and 60 can be moved axially with respect to distal tip 20 so as to vary the position of the distal ends of these core members with respect to distal tip 20. By doing so, the flexibility of the distal portion of wire guide 10 is varied. In another embodiment, the distal end of core member 40 is attached to distal tip 20 and cannot be moved axially with respect to distal tip 20. In yet another embodiment, the distal end of core member 40 is not attached to distal tip 20. In this embodiment, core members 40, 50 and 60 can be moved axially with respect to distal tip 20. A safety wire can be included in this embodiment to prevent excessive extension of coil 30.

FIG. 1(b) illustrates a partial view of another embodiment of wire guide 10. In this embodiment, band 70 is positioned around core members 40, 50 and 60 to hold the core members together within the lumen of coil 30 while allowing axial movement of the members in a proximal-distal direction. Of course, more than one band may be positioned along the length of the core members. FIG. 1(c) illustrates a partial view of yet another embodiment of wire guide 10. In this embodiment, core members 80, 90 and 100 are positioned within the lumen of coil 30. Core members 80 and 100 are tapered towards the distal end of wire guide 10, resulting in increased flexibility of the tapered portions of the members. The present embodiments include those in which all or some or none of the core members are tapered.

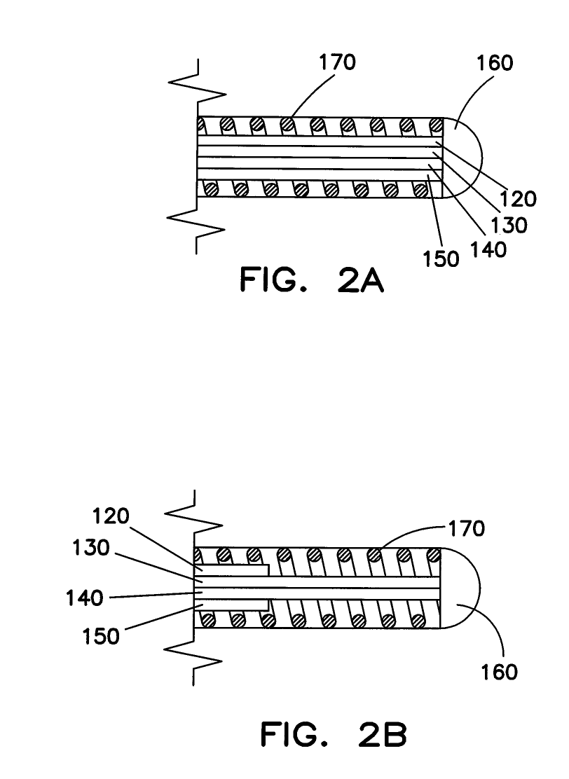

FIGS. 2(a) and 2(b) illustrate the distal portion of a wire guide including four core members 120, 130, 140 and 150 positioned within the lumen of coil 170. In FIG. 2(a) all four core members are positioned at their maximum distal positions against distal tip 160. In FIG. 2(b) core members 120 and 150 are moved proximally to increase the flexibility of the distal portion of the wire guide. Core members 130 and 140 remain at their maximum distal positions.

Control of the variation in flexibility of the wire guide may be increased by including additional core members within the coil of the wire guide. The present embodiments include wire guides having 2, 3, 4, 5, 6, 7 or more core members. In one embodiment, the core members are positioned side-by-side within the lumen of the wire guide coil. For the purposes of the present embodiments, core members are considered to be positioned side-by-side then they are bundled together in any configuration within the lumen of the coil.

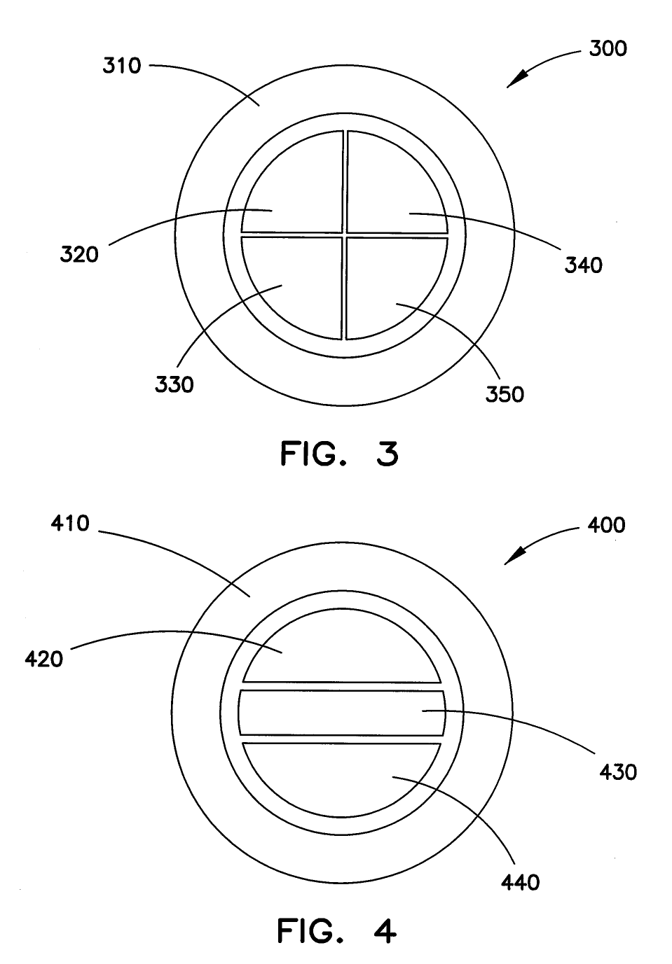

FIG. 3 illustrates a cross sectional view of one embodiment of wire guide 300 having four core members 320, 330, 340 and 350 positioned side-by-side within the lumen of coil 310. The cross section of each core member is in the form of a quarter segment of a circle. Of course, the present embodiments also include those having 2, 4, 5, 6, 7 or more core members each having a cross section of an appropriate segment of a circle. The core members may also have a cross section in the form of a transverse section of a circle. FIG. 4 illustrates a cross sectional view of one embodiment of wire guide 400 having three core members 420, 430 and 440 positioned side-by-side within the lumen of coil 410. Here, core members 420 and 440 each have a flat surface and a rounded surface and core member 430 has a first and a second flat surface. Each of the flat surfaces of core members 420 and 440 is positioned facing one of the flat surfaces of core member 430.

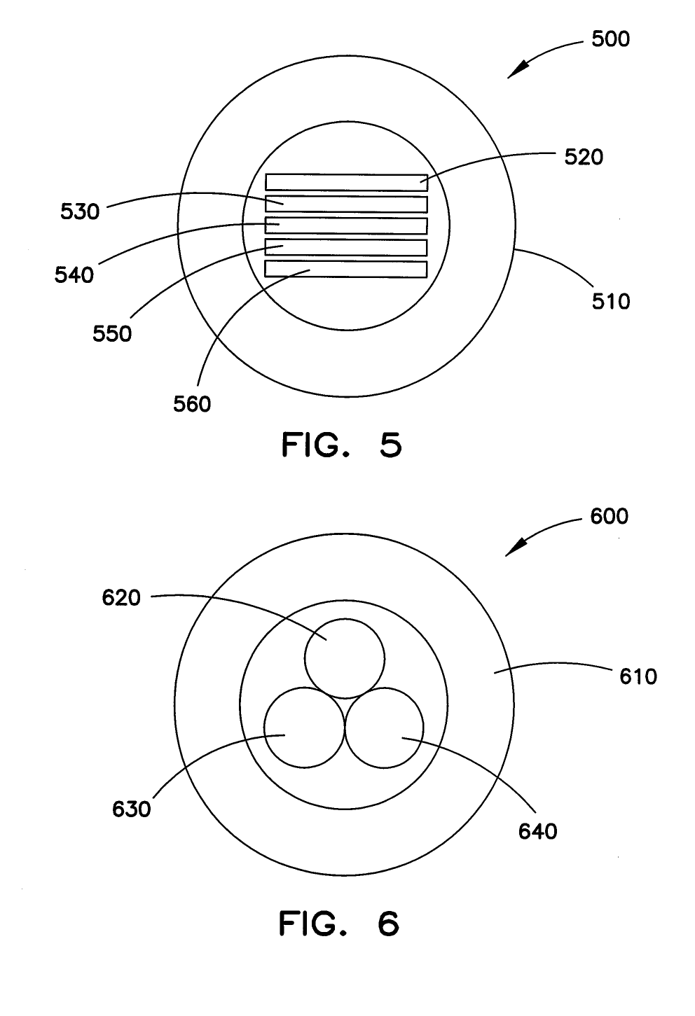

In other embodiments, the core members have a rectangular cross section. FIG. 5 illustrates a cross sectional view of wire guide 500 including five rectangular core members 520, 530, 540, 550 and 560 positioned side-by-side within the lumen of coil 510. The core members may also have a circular cross section. FIG. 6 illustrates a cross sectional view of wire guide 600 including three circular core members 620, 630 and 640 positioned side-by-side within the lumen of coil 610. In other embodiments, the core members may have a cross section that is, for example, square, triangular, elliptical or irregular. The core members positioned within the coil may have the same or different cross sectional shapes and many be formed from the same or different materials.

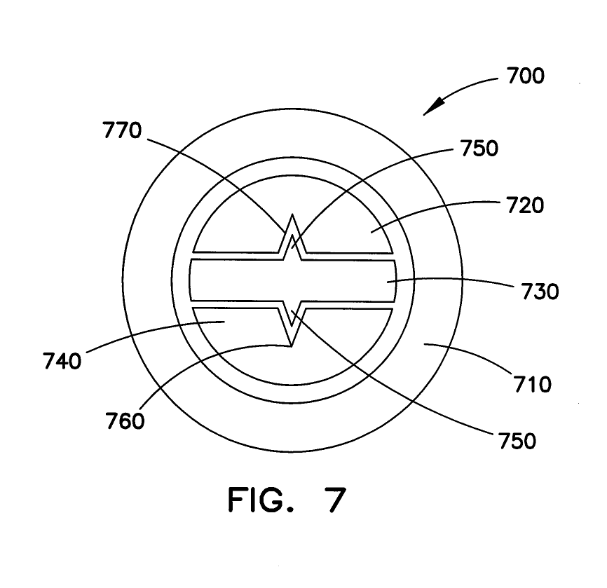

In another embodiment, at least one of the core members includes a protrusion shaped to engage an indentation in another core member. FIG. 7 illustrates one such configuration. Here, core members 720, 730 and 740 are positioned within the lumen of coil 710 of wire guide 700. Core member 730 includes two protrusions 750, one of which engages indentation 770 in core member 720 and the other of which engages indentation 760 in core member 740.

In certain embodiments, the core members are manufactured from a material such as stainless steel, a stainless steel alloy, platinum, palladium, a nickel-titanium alloy, such as NITINOL®, or combinations of these materials. Inclusion of a radiopaque material, such as platinum or gold, into the core members allows for better visibility during manipulation of the wire guide within the body of the patient. In certain embodiments, a radiopaque material is included in portions of one or more core members, for example, at the distal end of a core member. Where one or more of the core members are attached to distal tip of the wire guide, the attachment may be by methods including, but not limited to, adhesive, solder or laser welding.

The coil of the wire guide may be a single filar coil or a multifilar coil. Alternatively a cable tube may be used. The use of a multifilar coil or a cable tube may eliminate the need for a safety wire, allowing all of the space within the lumen of the coil to be occupied by the core members.

The coil may be formed from any suitable material including, but not limited to stainless steel, alloys including stainless steel, a nickel-titanium alloy, such as NITINOL®, or combinations of these materials. In certain embodiments, the wire guide includes a coating on at least a portion of the surface of the coil. The coating can include a material that reduces the coefficient of friction on that surface. For example, the coating may include a polymer such as, but not limited to, a fluoropolymer.

The external diameter of the coil and the number and shape of core members may be chosen to obtain a required wire guide size and flexibility. The dimensions given below are illustrative of some typical configurations and dimensions. In one embodiment, the external diameter of the coil is between 0.040 inches and 0.010 inches. In another embodiment, the external diameter of the coil is between 0.020 inches and 0.010 inches. In yet another embodiment, the external diameter of the coil is between 0.040 inches and 0.020 inches. In one embodiment the wire guide is formed from a coil having an external diameter of approximately 0.035 inches. In another embodiment, the wire guide is formed from a coil having an external diameter of approximately 0.015 inches.

The wire guide may also include a handle attached to the proximal end of the coil. In one embodiment the handle includes a plurality of adjustment members, one of which is attached to each of the movable core members. Each of the adjustment members can be moved axially and provides a means of varying the axial position of the attached core member. The handle may also include graduated markings indicating the axial position of the core members. A stop member limiting the extent of proximal movement of at least one of the core members may also be included.

Another aspect provides a method of varying the flexibility of wire guide while it is in place within the body of a patient. Referring again to FIGS. 2(a) and 2(b), distal tip 160 of the wire guide is inserted into a body lumen of a patient, for example, a vascular vessel. In one embodiment, the core members 120, 130, 140 and 150 of the wire guide are positioned at their maximum distal positions, i.e. in contact with distal tip 160. When the core members are so positioned, the distal portion of the wire guide is in its least flexible configuration. If there is a requirement to increase the flexibility of the distal portion of the wire guide, one or more of the core members are moved axially in a proximal direction and away from distal tip 160. For example, in FIG. 2(b), core members 120 and 150 have been moved proximally and away from distal tip 160.

Although the invention has been described and illustrated with reference to specific illustrative embodiments thereof, it is not intended that the invention be limited to those illustrative embodiments. Those skilled in the art will recognize that variations and modifications can be made without departing from the true scope and spirit of the invention as defined by the claims that follow. It is therefore intended to include within the invention all such variations and modifications as fall within the scope of the appended claims and equivalents thereof.

We claim:

1. A wire guide comprising:
a coil having a proximal end and a distal end and defining a lumen,
a distal tip positioned at the distal end of the coil,
a plurality of core members positioned side by side within the lumen,
wherein at least one of the plurality of core members is attached to the distal tip and extends from the distal tip to the proximal end of the coil.

2. The wire guide of claim 1, further comprising a band positioned around the plurality of core members and holding the plurality of core members together.

3. The wire guide of claim 1, wherein at least one of the plurality of core members is movable axially with respect to the coil, whereby the at least one of the plurality of core members can move proximally and away from the distal tip.

4. The wire guide of claim 1, wherein at least one of the plurality of core members comprises a square or rectangular cross section.

5. The wire guide of claim 1, further comprising a handle attached to the proximal end of the coil, wherein the handle comprises a plurality of adjustment members and wherein one of the plurality of adjustment members is attached to one of the plurality of core members and is movable axially to vary the axial position of the one of the plurality of core members.

6. The wire guide of claim 5, wherein the handle further comprises graduated markings, wherein the graduated markings indicate the axial position of the one of the plurality of core members.

7. The wire guide of claim 5, wherein the handle further comprises a stop member limiting the extent of proximal movement of at least one of the plurality of core members.

8. The wire guide of claim 1, wherein at least one of the plurality of core members is tapered towards a distal end.

9. The wire guide of claim 1, wherein at least one of the plurality of core members comprises a material selected from the group consisting of stainless steel, a stainless steel alloy, platinum, palladium, a nickel-titanium alloy and combinations thereof.

10. The wire guide of claim 1, comprising three core members, two of the core members having a flat surface and a rounded surface and one of the core members having a first flat surface and a second flat surface, wherein the flat surface of one of the core members having a flat surface and a rounded surface is positioned facing the first flat surface.

11. The wire guide of claim 1, wherein the coil is a multifialar coil.

12. The wire guide of claim 1, wherein at least one of the plurality of core members comprises a protrusion shaped to engage an indentation in another of the plurality of core members.

13. The wire guide of claim 1 having a variable flexibility dependent upon the relative axial positions of the plurality of core members.

14. The wire guide of claim 1 comprising at least three core members.

15. The wire guide of claim 14 comprising at least four core members.

16. The wire guide of claim 1, the coil having an external diameter between 0.040 inches and 0.010 inches.

17. A wire guide comprising:
a coil having a proximal end and a distal end and defining a lumen, the coil having an external diameter of between 0.040 inches and 0.010 inches,
a distal tip positioned at the distal end of the coil,
a plurality of core members positioned side by side within the lumen, and
a handle attached to the proximal end of the coil, wherein the handle comprises a plurality of adjustment members and wherein one of the plurality of adjustment members is attached to one of the plurality of core members and is movable axially to vary the axial position of the one of the plurality of core members.

18. A method of varying the flexibility of a wire guide positioned within a body lumen of a patient, the method comprising:
inserting a distal end of the wire guide into the body lumen, wherein the wire guide comprises:
a coil having a proximal end and a distal end and defining a lumen,
a distal tip positioned at the distal end of the coil,
a plurality of core members positioned side by side within the lumen, and
a handle attached to the proximal end of the coil, wherein the handle comprises a plurality of adjustment members and wherein one of the plurality of adjustment members is attached to one of the plurality of core members and is movable axially,
moving at least one of the adjustment members axially to vary the axial position of the one of the plurality of core members, whereby the flexibility of the wire guide is varied.
